(19) United States
(12) Patent Application Publication
(10) Pub. No.: US 2007/0224701 A1
(43) Pub. Date: Sep. 27, 2007

(54) Combination vertical and lateral flow immunoassay device

(75) Inventor: Robert W. Rosenstein, Ellicott City, MD (US)

Correspondence Address: DAVID W. HIGHET, VP AND CHIEF IP COUNSEL;BECTON, DICKINSON AND COMPANY, 1 BECTON DRIVE, MC 110, FRANKLIN LAKES, NJ, 07417-1880, US

(73) Assignee: Becton, Dickinson and Company, Franklin Lakes, NJ (US)

(21) Appl. No.: 11/355,669

(22) Filed: Feb. 16, 2006

(51) Int. Cl. G01N 33/558

(52) U.S. Cl. 436/514

(57) ABSTRACT

The invention relates to immunoassays that detect the presence or absence, or determine the amount present of a particular analyte. More specifically, the invention relates to multizone, multilayered immunoassay test devices that utilize a combination of vertical and lateral flow to detect an analyte of interest.

### FIELD OF THE INVENTION

The invention relates to immunoassays that detect the presence or absence, or determine the amount present of a particular analyte. More specifically, the invention relates to multizone, multilayered immunoassay test devices that utilize a combination of vertical and lateral flow to detect an analyte of interest.

### BACKGROUND OF THE INVENTION

In vitro diagnostic (IVD) tests have revolutionized the rapid analysis of analytes, and allow for a simple and cost effective detection method for a myriad of moieties including proteins such as enzymes and hormones, drugs and drug metabolites, antibodies, and nucleic acids. Many of these tests are based on immunoassays that combine the principles of chemistry and immunology to provide for quantitative and qualitative analyses of target analytes. The basic principle of these assays is the detection of an analyte-receptor reaction.

In recent years, immunoassays have evolved from expensive and complex procedures requiring calibrated machinery and skilled technicians for operation to more simplistic designs such as dip-sticks and test strips using relatively inexpensive binder support mediums that are easily operated by anyone because they only require the user to follow a simple series of directions. Today, immunoassays based on these binder support mediums offer rapid results and increasing sensitivity for the detection of a large number of analytes of interest. For example, the in-home use of immunoassay pregnancy tests that qualitatively detect the presence of human chorionic gonadotropin (hCG) in the urine is commonplace.

While there are many permutations to the design of the binder support medium based immunoassay, the basic design involves detecting the analyte of interest following its binding to a labeled receptor (or “tracer”), and the necessary separation of the free labeled receptor from the bound labeled receptor. The analyte of interest is generally contained or placed in a liquid sample that is then added to the immunoassay. As the liquid sample interacts with the active reagents in the immunoassay, immunological or chemical reactions may occur that ultimately allow for the detection of the presence of the analyte of interest.

One of the more common strategies utilized today for binder support medium based immunoassays is the lateral flow assay. The lateral flow assay is characterized as “lateral” because the flow of the liquid generally moves in a lateral direction relative to the device. The lateral direction of flow may alternatively be characterized as a length-wise or horizontal flow along the length of the device. The basic design of the lateral flow assay comprises the lateral flow of a liquid containing or lacking an analyte of interest across a porous membrane or series of juxtaposed porous membranes, at least one of which acts as a binder support medium. The liquid generally flows due to capillary action, or capillary flow, which is essentially the process by which water is drawn from a wet area in a medium and transported to a dry area through the pores of a material. Capillary action or flow is caused by capillary forces acting on the liquid such as adhesion, cohesion, and surface tension.

There are generally two types of assay formats utilized in the binder support medium devices. In a “sandwich” type assay, as the liquid sample flows across the device; the analyte, if present, binds with a receptor capable of detection, usually an antibody that recognizes the analyte of interest capable of being linked or bound to a label (or “tracer”) moiety that can be detected, either by the operator or by a machine. The labeled receptor is generally located within the membrane or one of the membranes of the immunoassay device. If the labeled receptor is contained in a separate component membrane from the binder support medium, the membrane containing the labeled receptor may be referred to as a tracer pad. If the labeled receptor is contained in the binder support medium, then the area of the binder support medium containing the labeled receptor may be referred to as a tracer zone.

As the analyte-labeled receptor complex flows laterally across the membrane or membranes via capillary action, it eventually comes into contact with a detection zone on the binder support medium of the immunoassay that comprises an immobilized capture receptor or ligand known as a binder, where it is bound. Excess liquid and unbound labeled receptor flow past the detection zone to an attached sump. The sump absorbs the liquid and prevents the liquid from flowing back across the membrane. The presence or absence of the analyte of interest is determined through inspection of the detection zone, where the presence of the analyte is indicated usually by a specific visually detectable signal.

In a competitive type assay, the receptor is generally the analyte itself, a homologue or derivative thereof, or a moiety that is capable of binding to an immobilized binder in the detection zone located in the binder support medium. As the liquid sample flows into the detection zone, the analyte contained in the liquid sample competes with the labeled receptor in binding the immobilized binder. The presence or absence in the sample of the analyte of interest is determined through inspection of the detection zone, wherein the greater the amount of analyte present in the liquid sample, the lesser the amount of labeled receptor bound in the detection zone.

Lateral flow devices that utilize various alterations of this general concept have been previously described. See, for example: U.S. Pat. No. 3,888,629; U.S. Pat. No. 4,632,901, U.S. Pat. No. 4,366,241; U.S. Pat. No. 4,943,522; U.S. Pat. No. 5,798,273; U.S. Pat. No. 5,607,863; U.S. Pat. No. 5,648,274; and U.S. Pat. No. 5,468,648. More complicated lateral flow devices include those described in, for example: U.S. Pat. No. 5,401,667; U.S. Pat. No. 4,855,240; U.S. Pat. No. 5,602,037; U.S. Pat. No. 5,712,170; U.S. Pat. No. 4,727,019; and U.S. Pat. No. 6,593,085.

Although the lateral flow concept has been widely utilized, lateral flow devices have a number of drawbacks. For example, accumulation of analyte-labeled receptor complex moieties can reduce the flow rate across the length of the membrane. Reduced flow rates on a membrane may result in a leading or lagging edge effect on the membrane that cause uneven distribution of the complexes across the detection zone. Such uneven distribution across the detection zone may lead to indicators in the detection zone that are difficult to read. In addition, the distance the analyte-labeled receptor complex is required to travel prior to contact with the immobilized binder results in an increased analysis time. Furthermore, many of the membranes utilized in lateral flow devices, such as nitrocellulose, may undesirably bind to the analyte of interest. This undesirable binding may result in a dilution of the analyte given the long distances required for the analyte to travel in a lateral device before encountering the detection zone. Dilution of the analyte can result in the reduced accuracy of the test device.

An alternative to the use of lateral flow devices includes utilizing vertical flow or “flow through” devices. In vertical flow devices, the liquid travels through vertically stacked or juxtaposed layers of membranes or other substances in a vertical direction relative to the device. The vertical direction may also be thought of as a thickness wise, or depth wise direction relative to the device. The analyte present in the liquid undergoes similar immunological and chemical reactions as described for the lateral flow devices. Examples of these types of devices are described in U.S. Pat. No. 5,073,484; U.S. Pat. No. 5,185,127; U.S. Pat. No. 5,654,162; U.S. Pat. No. 6,020,147; U.S. Pat. No. 6,106,732; and U.S. Pub. No. 2005/0124077.

One potential advantage of utilizing a vertical flow device may include the reduction in the leading edge effects seen in lateral flow devices. Another advantage may be the increased speed of the assay. In addition, the vertical flow device reduces the unwanted binding of the analyte to the membranes utilized in the assays, due to the shorter distance of travel through the membrane. However, traditional vertical flow devices required that the operator perform multiple steps in order to determine the presence or absence of an analyte. For example, some vertical flow devices require the operator to separately add sample, wash, and the tracer. Compared to lateral flow devices, the additional steps required of the operator with a vertical flow device can increase the error rate of the device. In addition, analyzing the results of a flow through device may be more difficult than a lateral device if multiple pads are utilized in the flow through device because the detection zone may not be easily accessible.

There is a need in the art for immunoassay devices that utilize the ease of use and detection of lateral flow devices with the speed and efficiency of vertical flow devices in a single device for detecting analytes of interest in a sample.

A further need exists for methods for detecting analytes of interest in a sample using an immunoassay device that incorporates both vertical and lateral flow strategies.

### SUMMARY OF THE INVENTION

The present invention can provide immunoassay devices, kits and methods for determining the presence or absence of an analyte of interest in a liquid sample utilizing a combination of vertical flow and lateral flow strategies. The invention can utilize a tracer pad comprising a labeled receptor that is vertically juxtaposed with a binder support medium. If the assay is a sandwich type assay, the receptor can be capable of binding to the analyte of interest. If the assay is a competitive type assay, then the receptor is generally not capable of binding to the analyte of interest, but can be capable of binding to a binder in a detection zone located on a binder support medium.

The tracer pad may comprise more than one labeled receptor, wherein each labeled receptor can be capable of binding to a different analyte of interest in a sandwich assay, or be capable of binding to a binder in a detection zone in a competitive assay. A liquid sample containing or lacking an analyte of interest can be applied to a specific location on the immunoassay device wherein the liquid sample wets the tracer pad. In certain exemplary embodiments, the liquid sample may be applied directly to the tracer pad. If the assay is a sandwich type assay and the analyte is present in the liquid sample, then the analyte can interact with the labeled receptor located in the tracer pad and form an analyte-labeled receptor complex. This analyte-labeled receptor complex can then flow vertically from the tracer pad to the binder support medium.

If the assay is a competitive type assay, then the analyte in the liquid sample does not bind with the receptor, but rather competes with the receptor in the detection zone to bind to a limited number of immobilized binders.

The binder support medium can be comprised of two portions. The first portion is the part of the binder support medium that is vertically juxtaposed with the tracer pad. This first portion is the portion of the binder support medium where at least one detection zone is located. The detection zone may be an area on the medium where a binder that recognizes an analyte-labeled receptor complex, receptor, analyte, or other moiety has been immobilized. If the assay is a sandwich type assay, as the liquid sample comes into contact with the detection zone after flowing vertically onto the binder support medium from the tracer pad, the analyte-labeled receptor complex, if present, may bind to the immobilized binder, and become trapped within the detection zone. If the assay is a competitive type assay, as the liquid sample comes into contact with the detection zone after flowing vertically onto the binder support medium from the tracer pad, the labeled receptor competes with the analyte of interest, if present, in binding the immobilized binder. Because the labeled receptor contains a label, the trapped complex in a sandwich assay, or the labeled receptor in a competitive assay, may be detected by the operator visually or using a machine that can analyze the label on the labeled receptor in the detection zone.

The second portion of the binder support medium is that part of the binder support medium that is not vertically juxtaposed with the tracer pad. This portion of the binder support medium may allow the liquid sample to laterally flow away from the detection zone. Labeled receptor moieties that are not bound to the analyte of interest or the immobilized binder may be cleared from the detection zone through the lateral flow of the liquid sample across the second portion of the binder support medium. This clearance of the unbound labeled receptor allows for the generation of a more accurate indication, eliminating or reducing the unbound labeled receptor from remaining in the detection zone.

The second portion of the binder support medium may comprise a control zone. The control zone can comprise an indicator that allows one to determine if the liquid sample has migrated to the control zone. For example, the control zone may comprise an anhydride moiety that turns a certain color on contact with the liquid sample. Because the detection zone precedes the control zone in the flow path of the liquid sample, analyzing the control zone for an indication of liquid sample contact indicates that the liquid sample has also made contact with the detection zone.

Furthermore, in certain exemplary embodiments, the control zone may indicate whether the assay had worked by utilizing components in the control zone that indicate certain physiological conditions. For example, in certain exemplary embodiments, the control zone may comprise immobilized analyte that binds to unbound labeled receptor. As the liquid sample passes through the control zone, the immobilized binder binds to the unbound labeled receptor, indicating that the physiological conditions of the assay were in an adequate workable range for binding.

After the liquid sample has cleared the detection zone in the first portion of the binder support medium, the operator can observe the result of the assay. If the assay is one with a visually detectable label, then the operator can simply look at the detection zone area to analyze the result. Generally, if the analyte of interest is present in the liquid sample, then an analysis of the detection zone will signal this.

The immunoassay device may further comprise additional elements. For example, a sample pad may be vertically juxtaposed with the tracer pad. A liquid sample may be applied to the sample pad where it then flows vertically from the sample pad to the tracer pad, and from the tracer pad to the binder support medium.

In addition, a sump may be laterally juxtaposed with the binder support medium. The sump is capable of absorbing excessive liquid applied to the device as the liquid flows laterally from the first portion of the binder support medium to the second portion of the binder support medium. The sump absorbs the liquid as the liquid flows to the lateral end of the second portion of the binder support medium.

The immunoassay device may comprise spacer pads placed between the various other pads. For example, a spacer pad may be placed between the sample pad and tracer pad, and/or the tracer pad and binder support medium.

The device may further comprise a supportive housing. This allows the device to be easily handled, prevents disturbance of the assay during use, and protects the components of the assay during shipment. The housing may comprise a window or aperture for applying the liquid sample, as well as a window or aperture for viewing the detection and control zones.

The invention is not limited to the type of assay employed. For example, the assay may be a sandwich type assay or a competitive type assay, or other assay formats known to one of ordinary skill in the art. In addition, the invention is not limited to they type of analysis. For example, the analysis of the analyte may be qualitative, semi-quantitative, or quantitative. Qualitative, semi-quantitative, and quantitative assays are known to one of ordinary skill in the art. Semi-quantitative or quantitative analysis of the concentration or amount of an analyte of interest in a liquid sample requires the use of a calibration mechanism. For example, the calibration mechanism may be a known amount of analyte. The calibrated analyte may be placed on the binder support medium in a separate zone (calibration zone) from the detection zone, wherein the tracer pad comprises an excess of labeled receptor that is capable of binding to the analyte in the calibration zone. To determine semi-quantitative or quantitative amounts, the intensity of label in the detection zone is compared to the intensity of label in the calibration zone. Such calibration mechanisms are generally known by one of ordinary skill in the art.

The invention provides a shorter distance an analyte must travel to encounter a detection zone because the liquid sample flows through the various pads to the detection zone in a vertical fashion as opposed to a lateral fashion. This may result in faster assays comprising less materials and a smaller size. Economical saving may be realized due to the reduced materials and smaller size, as well as potentially resulting in easier manufacturing. Because of the vertical aspects of the assay, the liquid sample may be exposed to a greater amount of tracer and binder, without experiencing the leading edge effects seen in strictly lateral flow assays. The invention can provide for a lateral flow away from the detection zone, and can result in an easily discernable indication from the detection zone as compared to vertical flow devices.

### BRIEF DESCRIPTION OF THE FIGURES

Specific exemplary embodiments of the present invention will now be described by way of example only and with reference to the accompanying drawings.

FIG. 1 is a schematic of the immunoassay device according to one exemplary embodiment of the invention wherein a detection zone is located within a first portion of the binder support medium, and a control zone is located within a second portion of the binder support medium, the second portion being downstream from the area of contact of the binder support medium with the tracer pad.

FIG. 2 is a schematic of the immunoassay device according to one exemplary embodiment of the invention wherein the detection zone is analyzed for the presence of a detectable indicator depicting the presence of an analyte of interest.

FIG. 3 is a schematic of the immunoassay device according to one exemplary embodiment of the invention wherein the assay is illustrative of a competitive assay.

FIG. 4 is a schematic of the immunoassay device according to one exemplary embodiment of the invention wherein the assay is illustrative of a competitive assay.

FIG. 5 is a schematic of the immunoassay device according to one exemplary embodiment of the invention wherein the tracer pad further comprises a control labeled receptor capable of binding a ligand immobilized in the control zone.

FIG. 6 is a schematic of the immunoassay device according to one exemplary embodiment of the invention further comprising a sump laterally juxtaposed to the binder support medium.

FIG. 7 is a schematic of the immunoassay device according to one exemplary embodiment of the invention wherein a sample pad is vertically juxtaposed to a tracer pad, the tracer pad further being vertically juxtaposed with the binder support medium.

FIG. 8 is a schematic of the immunoassay device according to one exemplary embodiment of the invention wherein a sample application member is in fluid flow contact with a sample pad.

FIG. 9 is a schematic of the immunoassay device according to one exemplary embodiment of the invention wherein a sample pad is vertically juxtaposed to a tracer pad, the tracer pad being vertically juxtaposed with the binder support medium, and a sump is laterally juxtaposed with the binder support medium.

FIG. 10 is a schematic of the immunoassay device according to one exemplary embodiment of the invention wherein an additional detection zone is located in the second portion of the binder support medium.

FIG. 11 is a schematic of the immunoassay device according to one exemplary embodiment of the invention wherein a control zone is located in the first portion of the binder support medium.

FIG. 12 is a schematic of the immunoassay device according to one exemplary embodiment of the invention wherein the sump surrounds the binder support medium and is laterally juxtaposed with the binder support medium.

FIG. 13 is a schematic of the immunoassay device according to one exemplary embodiment of the invention wherein the immunoassay device further comprises a supportive housing.

FIG. 14 is a schematic of the immunoassay device according to one exemplary embodiment of the invention wherein spacer pads have been inserted in between the sample pad and tracer pad, as well as the tracer pad and the binder support medium.

FIG. 15 is a schematic of a flow diagram according to one exemplary embodiment of the invention wherein a method for detecting an analyte of interest is illustrated.

FIG. 16 is an overview of a representative schematic of a constructed immunoassay device.

### DETAILED DESCRIPTION

The present invention can provide immunoassay devices, kits and methods for determining the presence of an analyte of interest in a liquid sample utilizing a combination of vertical flow and lateral flow strategies.

The terms “vertical” or “vertically” as used herein generally means parallel to the thickness or depth, as opposed to the length and width dimensions of the elements utilized in the device, such as the pads or mediums. The term “lateral” or “laterally” as used herein generally means parallel to the length, as opposed to the width and depth dimensions of the elements utilized in the device, such as the pads and mediums. In many embodiments, the elements are substantially planar and have a length, or lateral dimension, that is greater than the thickness, or vertical dimension. However, it is recognized that the magnitudes of these dimensions relative to each other can be changed without departing from the scope and spirit of the invention.

Generally, the terms “vertical,” “vertically,” “lateral,” and “laterally” also can describe the juxtaposition or orientation of the elements of the device. For vertically juxtaposed elements, a line normal to and intersecting the planar surface of one such element also will be substantially normal to and intersect the planar surface of the other vertically juxtaposed elements. It is further recognized that gravity is not necessary for the operation of the device because the liquid sample may flow by capillary flow or action through the pads and mediums of the device.

In particular, the invention can provide a device comprising:
a) a tracer pad comprising a receptor moiety attached to a tracer or label moiety, wherein the tracer pad is capable of accepting a liquid sample; and
b) a binder support medium comprising at least one detection zone for the detection of an analyte of interest, derivative, or homologue thereof, the binder support medium comprising at least a first portion and a second portion;
   wherein the first portion of the binder support medium is vertically juxtaposed to the tracer-pad, the first portion containing at least one detection zone, and the second portion of the binder support medium is not vertically juxtaposed with the tracer pad, the second portion providing lateral flow access for the liquid sample away from the first portion. In exemplary embodiments, the immunoassay device can further comprise a sample pad vertically juxtaposed to the tracer pad, the sample pad being capable of accepting a liquid sample. In additional exemplary embodiments, the immunoassay device can further comprise a sump laterally juxtaposed to the binder support medium. In a further exemplary embodiment, the immunoassay device can further comprise a housing to provide support for the device. Preferably, the juxtaposition is such that the liquid sample can flow from one component to the next.

The invention can allow for a liquid sample containing or lacking an analyte of interest to be applied to the device and flow vertically from the tracer pad to a first portion of a binder support medium comprising a detection zone. The first portion of the binder support medium is vertically juxtaposed to the tracer pad. The liquid sample can then flow laterally away from the first portion of the binder zone to a second portion of the binder support medium. The second portion of the binder support medium is not vertically juxtaposed with the tracer pad. In one exemplary embodiment, the liquid sample flows away from the detection zone on the first portion of the binder support medium to the second portion of the binder support medium, and then to a sump laterally juxtaposed with the binder support medium.

The invention can maintain the accuracy and sensitivity of analyte detection while providing for an improved flow rate, increased speed of detection due to the sample flowing vertically across thin pads in sequence, and reduced clogging and filtering problems associated with strictly lateral flow devices. Furthermore, the invention can allow for the production of smaller immunoassay devices and the use of less materials in producing such devices. In addition, by using a vertical flow into the detection zone, the detection zone can be expanded, reducing leading edge effects often seen in lateral flow devices which may result in difficulties in detecting the presence or absence of the analyte of interest.

In certain exemplary embodiments of the invention, an immunoassay device can be provided comprising:
a) a tracer pad comprising a labeled receptor, wherein the tracer pad is capable of directly accepting a liquid sample, and serves as the entry point for the liquid sample; and
b) a binder support medium comprising at least one detection zone for the detection of an analyte of interest, derivative, or homologue thereof, the binder support medium comprising at least a first portion and a second portion;
   wherein the first portion of the binder support medium is vertically juxtaposed to the tracer pad, the first portion containing at least one detection zone, and the second portion of the binder support medium is not vertically juxtaposed with the tracer pad, the second portion providing lateral flow access for the liquid sample away from the first portion. In one particular exemplary embodiment, the device further comprises c) a sump laterally juxtaposed to the binder support medium. Preferably, the juxtaposition is such that the liquid sample can flow from one component to the next.

In certain exemplary embodiments of the invention, an immunoassay device can be provided comprising:
a) a sample pad capable of accepting a liquid sample, which serves as the entry point for the liquid sample;
b) a tracer pad comprising a labeled receptor, wherein the tracer pad is vertically juxtaposed below the sample pad; and
c) a binder support medium comprising at least one detection zone for the detection of an analyte of interest, derivative, or homologue thereof, the binder support medium comprising at least a first portion and a second portion;
   wherein the first portion of the binder support medium is vertically juxtaposed to the tracer pad, the first portion containing at least one detection zone, and wherein the second portion of the binder support medium is not vertically juxtaposed with the tracer pad, the second portion providing lateral flow access for the liquid sample away from the first portion. In one exemplary embodiment, the device further comprises d) a sump laterally juxtaposed to the binder support medium. Preferably, the juxtaposition is such that the liquid sample can flow from one component to the next.

In one exemplary embodiment of the invention, the binder support medium comprises more than one detection zone, wherein at least one detection zone is vertically juxtaposed with a tracer pad. In one exemplary embodiment, in addition to the detection zone vertically juxtaposed with the tracer pad, at least one detection zone is located downstream of the vertically juxtaposed detection zone. In one exemplary embodiment, the downstream detection zone can be an additional detection zone for the same analyte of interest as that of the detection zone vertically juxtaposed with the tracer pad, or it can be a detection zone for a different analyte. In an additional exemplary embodiment, the binder support medium can further comprise a control zone, wherein the quality of the assay can be ascertained therefrom.

In certain exemplary embodiments of the invention, the immunoassay optionally comprises a housing, wherein the various aspects of the invention are contained therein. The housing of the invention can comprise apertures that can allow access to the sample pad or tracer pad, where applicable, for the application of the liquid sample. In addition, the housing can comprise apertures in direct relation with the detection zones and control zones, wherein a user can analyze the detector zone and control zone through the apertures to determine if an indicator is present signaling the presence or absence of the analyte of interest in the sample, and whether the assay was viable as indicated by the control. In certain exemplary embodiments of the invention, a spacer consisting of, for example, but not limited to, a non-reactive, porous material such as paper, cellulosic membranes, nitrocellulose, nylon, glass fiber, Dacron, or rayon may be inserted between any of the elements of the invention. Preferably, the insertion is such that the liquid sample is allowed to flow from one component to the next.

Utilization of the invention can allow for a liquid sample containing or lacking an analyte of interest to be applied to a tracer pad, the tracer pad comprising a mobile labeled receptor comprising i) a label moiety capable of detection attached to ii) a receptor. The receptor utilized in the invention may be dependent on the type of assay desired. In a sandwich type assay, the receptor may be capable of binding to the analyte of interest, derivative, or homologue thereof. In a competitive type assay, the receptor may not be capable of binding to an analyte of interest, derivative, or homologue thereof, but rather competes with the analyte for binding to the immobilized binder in the detection zone. Alternatively, the liquid sample can be applied to a sample pad that is vertically juxtaposed with the tracer pad, wherein the liquid sample flows vertically from the sample pad to the tracer pad. The sample can then flow vertically through the tracer pad, wherein, if the assay is a sandwich type assay, the labeled receptor can be bound to the analyte of interest, if present, and can form a mobile analyte-labeled receptor complex. The liquid sample, after passing through the tracer pad, may flow vertically to a binder support medium. The binder support medium comprises an immobilized binder in a detection zone that is capable of binding the analyte-labeled receptor complex, if a sandwich type assay, or the labeled receptor and analyte of interest if a competitive type assay, or the unbound labeled receptor, wherein the labeled receptor is a capable of binding the analyte of interest. The detection zone may allow for the concentration of the analyte-labeled receptor complex or the labeled receptor, wherein the label provides an indication of the presence or absence of the analyte of interest.

The liquid sample can flow vertically from the tracer pad to the detection zone located on the first portion of the binder support medium, wherein the analyte-labeled receptor complex if a sandwich type assay, or the analyte and labeled receptor if a competitive type assay, can bind to the immobilized binder contained in the detection zone. The portion of the liquid sample that is not bound by the immobilized binder can flow laterally to a second portion of the binder support medium, wherein the second portion is not vertically juxtaposed with the tracer pad. In certain exemplary embodiments, the liquid sample can flow laterally to a sump pad laterally juxtaposed with the binder support medium. In one exemplary embodiment, the sump pad can surround the binder support medium, allowing for multi-directional flow away from the first portion of the binder support medium to the second portion of the binder support medium, and then to the sump.

The term “immunoassay,” as used herein, generally includes assays involving at least one pair of moieties that interact by means of a specific binding interaction that is dependant on the physical and/or chemical characteristics of the moieties involved, and is not necessarily limited to assays in which the moieties are antibodies.

The invention can be utilized to analyze an analyte of interest in a liquid sample. The term “analyte” generally refers to the compound, composition, moiety, aggregation, or other substance to be detected. The analyte can be any substance for which there exists a mechanism for detecting the substance utilizing an analyte-ligand interaction. For example, the analyte, or portion thereof, can be an antigen or hapten having at least one determinant site, wherein a naturally occurring or synthetically derived antibody binds thereto.

Analytes that can be analyzed utilizing the invention include, but are not limited to, toxins, organic compounds, proteins, peptides, microorganisms, bacteria, viruses, amino acids, nucleic acids, carbohydrates, hormones, steroids, vitamins, drugs (including those administered for therapeutic purposes as well as those administered for illicit purposes), pollutants, pesticides, and metabolites of or antibodies to any of the above substances. The term analyte may also include any antigenic substances, haptens, antibodies, macromolecules, aggregated, or combinations thereof.

The term “sample” generally refers to anything that may contain an analyte for which an analyte assay is desired. The sample may be a biological sample, such as a biological fluid or a biological tissue. Examples of biological fluids include urine, blood, plasma, serum, saliva, semen, stool, sputum, cerebral spinal fluid, tears, ocular lens fluid, sweat, milk, ascites fluid, synovial fluid, peritoneal fluid, transdermal exudates, pharyngeal exudates, bronchoalveolar lavage, tracheal aspirations, cerebrospinal fluid, cervical mucus, vaginal or urethral secretions, mucus, amniotic fluid or the like. Fluid homogenates of cellular tissues such as, for example, hair, skin and nail scrapings, meat extracts and skins of fruits and nuts can also be considered biological fluids.

Biological tissues can be single cells or aggregates of cells, usually of a particular kind together with their intercellular substance that form one of the structural materials of a human, animal, plant, bacterial, fungal or viral structure, including connective, epithelium, muscle and nerve tissues. Examples of biological tissues include organs, tumors, lymph nodes, arteries and individual cell(s).

Besides biological fluids, other samples can be used such as water, food products, soil extracts, and the like for the performance of industrial, environmental, or food production assays as well as diagnostic assays. In addition, a solid material suspected of containing the analyte can be used as the test sample once it is modified to form a liquid medium or to release the analyte into a liquid medium.

“Liquid sample” refers to a material suspected of containing an analyte(s) of interest, which material has sufficient fluidity to flow through an immunoassay device in accordance herewith. The liquid sample can be used as obtained directly from the source or following a pretreatment so as to modify its character. Such samples can include human, animal or man-made samples. The sample can be prepared in any convenient medium which does not interfere with the assay. Typically, the sample can be an aqueous solution or biological fluid.

The liquid sample can be treated prior to its application on the immunoassay device. Treatment, if necessary, may involve preparing plasma from blood, diluting viscous fluids, and the like. Methods of treatment can involve, but are not be limited to, filtration, distillation, separation, concentration, inactivation of interfering components, and the addition of reagents. Methods utilized for the selection and pretreatment of biological, industrial, and environmental samples prior to testing are generally known by one of ordinary skill in the art.

In one aspect of the invention, a method for determining the presence or absence of an analyte of interest in a liquid sample utilizing vertical and lateral flow of the liquid sample is provided, the method comprising:
a) applying a liquid sample to an assay device, wherein the liquid sample flows:
   i. vertically to a first portion of a binder support medium from a tracer pad comprising a labeled receptor, the first portion of the binder support medium being vertically juxtaposed to the tracer pad, and the first portion comprising at least one detection zone; and,
   ii. laterally from the first portion of the binder support medium to the second portion of the binder support medium, wherein the second portion of the binder support medium is not vertically juxtaposed with the tracer pad,
b) determining the presence or absence of the analyte in the liquid sample by analyzing the detection zone.

Referring now to the drawings, in which like numerals represent like elements throughout the several Figures, aspects of the invention and the illustrative operating environment will be described. The Figures, while representative of certain exemplary embodiments of the invention, are not intended to limit the invention in any way.

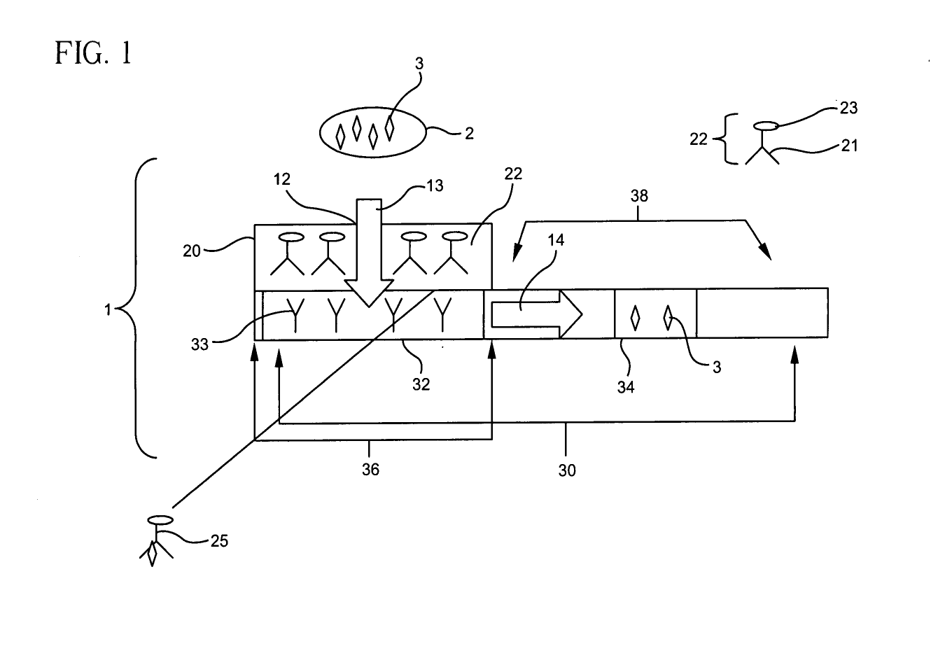

FIG. 1 illustrates a schematic of one exemplary embodiment of an immunoassay device 1 that can be utilized to detect the presence of an analyte of interest 3 in a liquid sample 2. The device 1 comprises a tracer pad 20. The tracer pad 20 can be comprised of a porous material, such as, for example, high density polyethylene sheet material, non-woven spun-laced acrylic fiber, polyvinyl chloride, polyvinyl acetate, copolymers of vinyl acetate and vinyl chloride, polyamide, polycarbonate, polystyrene, untreated paper, cellulose blends, cellulose derivatives such as cellulose acetate and nitrocellulose, fiberglass, cloth including natural and synthetic cloths, polyester, an acrylonitrile copolymer, Rayon, glass fiber, porous gels such as silica gel, agarose, dextran, and gelatin; porous fibrous matrixes; starch based materials, such as Sephadex®. brand cross-linked dextran chains; ceramic materials; films of polyvinyl chloride and combinations of polyvinyl chloride-silica; and the like.

The tracer pad 20 material can be treated with blocking and stabilizing agents. Blocking agents include bovine serum albumin (BSA), methylated BSA, casein, nonfat dry milk. Stabilizing agents are readily available and are known by those of ordinary skill in the art. Stabilizing agents may be used, for example, to stabilize labeled receptor reagents. For example, employment of blocking and stabilizing agents together with labeled receptor reagents in the tracer pad followed by the drying of the blocking and stabilizing agents (e.g., a freeze-drying or forced air heat drying process) can be utilized to attain improved performance of the device.

The tracer pad 20 further comprises a labeled receptor 22. The labeled receptor 22 can be comprised of i) a receptor 21, and ii) a detectable label 23 that serves as a reporter or tracer moiety attached to the receptor. The attachment may be by covalent or non-covalent binding, but the method of attachment may not be important to the invention. The label 23 allows the analyte specific labeled receptor 22 to be detected in the detection zone 32.

The invention is not dependent on the particular assay format selected, however, the receptor 21 portion of the labeled receptor 22 may be dependent upon the assay format selected. For example, as indicated in FIG. 1, the format may be a sandwich type of assay, and the receptor 21 portion of the labeled receptor 22 can be capable of specifically binding or complexing with the analyte 3. For example, the analyte 3 may be an antigen, wherein an antibody specific for the antigen may be used as the receptor 21, or immunologically reactive fragments of the antibody, such as F(ab′)2, Fab or Fab′ may be used as the receptor 21. These receptors 21 coupled to the label 23 can then bind to an analyte of interest 3 if present in the sample as the sample passes through the tracer pad 20, and form an analyte-labeled receptor complex 25. These analyte-labeled receptor complexes 25 can then be carried into the detection zone 32 on the binder support medium 30 by the vertical fluid flow 13 through the device 1. When the analyte-labeled receptor complex 25 reaches the detection zone 32, it can be captured by an immobilized binder 33.

The receptor 21 may be a specific binding moiety for the analyte 3. The most common types of analyte-labeled receptor complexes 25 are antigen-antibody complexes. For example, the analyte of interest 3 can be an antigen, and the receptor 21 an antibody Alternatively, the analyte of interest 3 can be an antibody, and the receptor 21 an antigen. The receptor 21 may also be a specific binding moiety for an aggregation of moieties; for example, an antibody raised against an immune complex of a second antibody and its corresponding antigen may be considered to be a receptor 21 for the immune complex.

Other analyte-labeled receptor complexes 25 capable of forming in the invention include specific binding pairs such as biotin and avidin, streptavidin and anti-biotin, carbohydrates and lectins, complementary nucleotide sequences, complementary peptide sequences, effector and receptor molecules, enzyme cofactors and enzymes, enzyme inhibitors and enzymes, a peptide sequence and an antibody specific for the sequence or the entire protein, polymeric acids and bases, dyes and protein binders, peptides and specific protein binders, for example, ribonuclease, S-peptide and ribonuclease S-protein, metals and their chelators, aptamers and their associated binding partner, complementary nucleic acid sequences, and the like.

The receptor 21 can be, for example, an immuno-reactant such as an antibody, antigen, hapten, or complex thereof. Antibodies useful as receptors 21 in the invention include those specifically reactive with various analytes. If the receptor 21 is an antibody, particularly useful antibodies for use in the invention include IgG or IgM antibodies or mixtures thereof, which are essentially free of association with antibodies capable of binding with non-analyte moieties. The antibodies may be polyclonal or monoclonal and can be commercially available or may be obtained by mouse ascites, tissue culture or other techniques known to one of ordinary skill in the art. The use of mixtures of monoclonal antibodies of differing antigenic specificities or of monoclonal antibodies and polyclonal antibodies may be desired. Fragments of antibody molecules may also be used as specific binding receptors 21 according to the invention including half antibody molecules and Fab, Fab′ or F(ab′)2 fragments known to one of ordinary skill in the art. Regardless of the particular source or type of antibodies, however, it is preferred that they be generally free of impurities. The antibodies may be purified by column chromatography or other conventional means but are preferably purified according affinity purification techniques known to one of ordinary skill in the art.

The receptor 21 may also be an antigen or a hapten, whether natural or synthesized, which present antigenic determinants for which the analyte of interest 3: can specifically react with. Synthesized antigens include those which are constructed according to conventional chemical syntheses as well as those constructed according to recombinant DNA techniques. The receptor 21 may be a nucleic acid sequence capable of binding to a complementary sequence, or in the case of an apatamer, a peptide, protein, or other moiety.

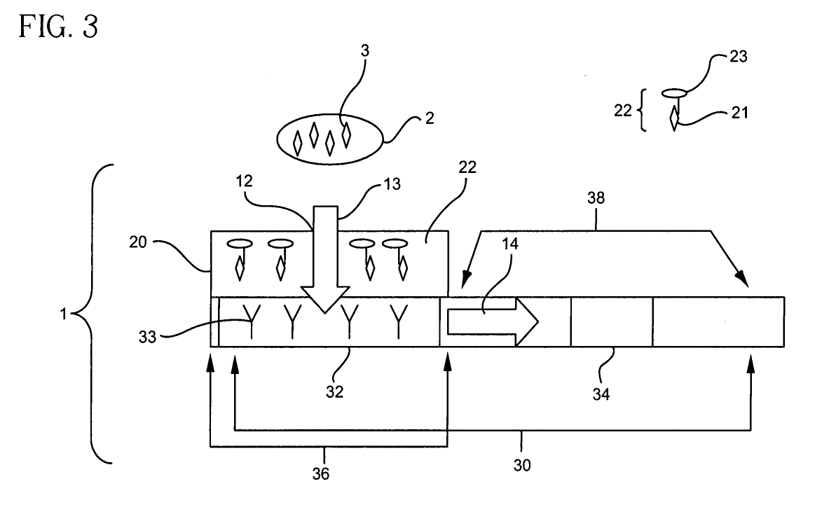

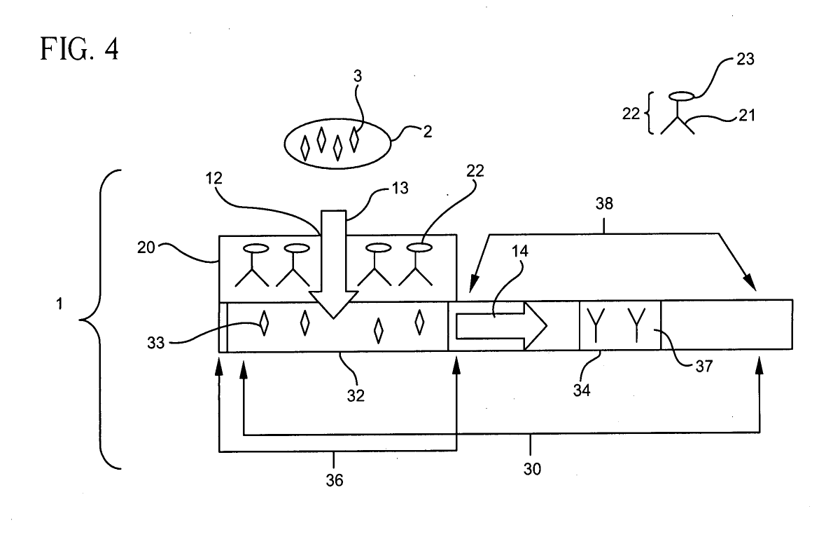

In an alternative exemplary embodiment, the assay may be a competitive assay, and the receptor 21 component may be a receptor that does not bind to an analyte 3, but rather is capable of competing with the analyte 3 for the immobilized binder 33. In certain exemplary embodiment, the receptor 21 may be the analyte of interest 3, a homologue, or a derivative thereof. This strategy is further illustrated in FIG. 3. In a further alternative embodiment, the assay may be a competitive assay, wherein the receptor 21 may be an antibody to the analyte of interest 3, a homologue, or derivative thereof, and the immobilized binder 33 may be the analyte of interest 3, wherein the analyte of interest 3 competes with the immobilized binder 33. This strategy is further illustrated in FIG. 4.

The label 23 of the analyte specific labeled receptor 22 can be any one of a wide variety of detectable labels known to one of ordinary skill in the art. The label 23 attached to the receptor 21 can be any substance which is capable of detection by visual or instrumental means. Various labels 23 suitable for use in the invention include labels which are detectable through either chemical or physical means. Such labels 23 can include, but are not limited to, enzymes and substrates, chromogens, catalysts, fluorescent compounds, chemiluminescent compounds, and radioactive labels. Other suitable labels 23 include particulate labels such as colloidal metallic particles such as gold, colloidal non-metallic particles such as selenium or tellurium, dyed or colored particles such as a dyed plastic or a stained microorganism, colored organic polymer latex particles and liposomes, colored beads, polymer microcapsules, sacs, erythrocytes, erythrocyte ghosts, or other vesicles containing directly visible substances, and the like. Typically, a visually detectable label can be used as the label 23 component of the labeled receptor 22, thereby providing for the direct visual or instrumental readout of the presence or amount of the analyte in the liquid sample without the need for additional detectable components at the detection zone 32. For example, the labels 23 can be moieties which are visible when present in sufficient quantity in the detection zone 32. Alternatively, the label can be detected with an instrument. For example, the label 23 can be with the aid of an optical filter and/or applied stimulation, for example, UV light to promote fluorescence.

Indirect labels, such as enzymes, e.g. alkaline phosphatase and horseradish peroxidase, can also be used as a label 23 but these usually require the addition of one or more developing reagents such as substrates before a visible indicator can be detected. Such additional reagents can be incorporated in the tracer pad 20. Alternatively, the developing reagents can be added to the liquid sample 2 before contact with the tracer pad 20, or the tracer pad 20 can be exposed to the developing reagents after the binding reaction between the analyte 3 and labeled receptor 22 has taken place.

The tracer pad 20 can also comprise stabilizers, buffers, surfactants and other agents which improve the performance of the assay. The tracer pad 20 may include components that assist in the transfer of the liquid sample 2 through the assay device. The functions of the tracer pad 20 may further include, for example: pH control/modification and/or specific gravity control/modification of the liquid sample 2 applied, removal or alteration of components of the liquid sample 2 which may interfere or cause non-specific binding in the assay, or to direct and control liquid sample flow to the detection zone 32.

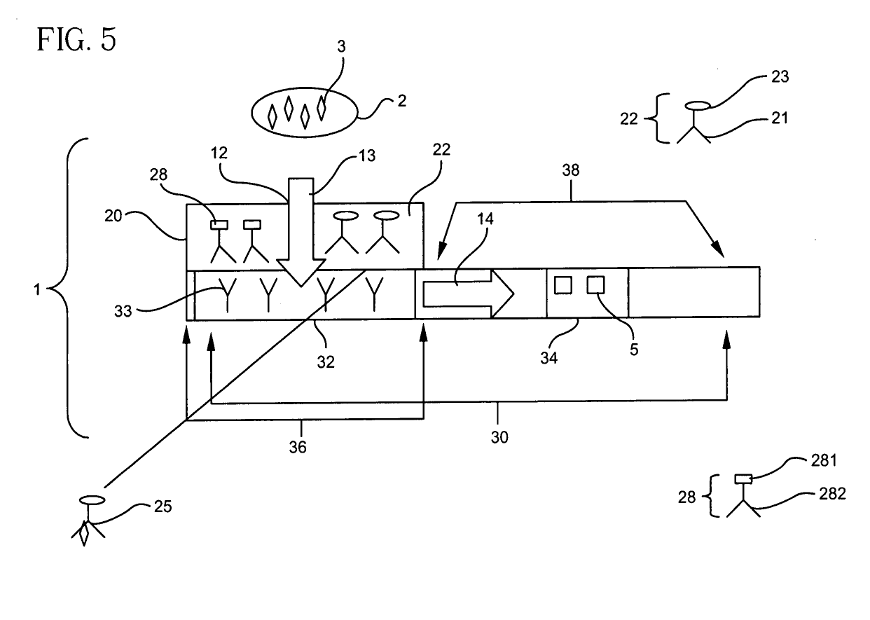

The tracer pad 20, in certain exemplary embodiments, may further comprise a labeled receptor specific for a ligand immobilized in the control zone 34. Such an embodiment is further exemplified in FIG. 5.

The liquid sample 2 can be added to the tracer pad 20 at an application point 12. The liquid sample 2 can then flow vertically 13 from the tracer pad 20, where it can interact with the labeled receptor 22 forming an analyte-labeled receptor complex 25, to the binder support medium 30.

The tracer pad 20 is vertically juxtaposed to a binder support medium 30, as illustrated in FIG. 1. The binder support medium 30 comprises a first portion 36 and a second portion 38. The first portion 36 of the binder support medium 30 comprises that portion of the binder support medium 30 that is vertically juxtaposed to the tracer pad 20. The first portion 36 of the binder support medium 30 comprises a detection zone 32 for analyzing the analyte of interest 3. Liquid sample 3 can flow vertically 13 from the tracer pad 20 onto the first portion 36 of the binder support medium 30, where it can interact with the detection zone 32.

The second portion 38 of the binder support medium 30 comprises that portion of the binder support medium 30 that is not vertically juxtaposed to the tracer pad 20, or what can be characterized as an open region. The second portion 38 of the binder support medium 30 may further comprise a control zone 34. Following interaction with the detection zone 32 in the first portion 36 of the binder support medium 30, the liquid sample 2 can flow laterally away from the detection zone 32 to the second portion 38 of the binder support medium 30.

The binder support medium 30 is vertically juxtaposed with the tracer pad 20 at the contact point on the first portion 36 of the binder support medium 30. Liquid sample 2 flows vertically 13 through the tracer pad 20 to the first portion 36 of the binder support medium 30, and then laterally from the first portion 36 to the second portion 38 of the binder support medium 30.

The binder support medium 30 that can be utilized in the invention include, but are not limited to, substrate materials having capillarity and the capacity for chromatographic solvent transport of non-immobilized reagents and reactive sample components by means of a selected chromatographic solvent. The binder support medium 30 of the assay device of the invention can be any suitably absorbent, porous or capillary possessing material through which a solution containing the analyte can be transported by a wicking action.

The particular dimensions of the binder support medium 30 will be a matter of convenience, depending upon the size of the liquid sample 2 involved, the assay protocol, the means for detecting and measuring the label 23, and the like. The binder support medium 30 used with the invention can be in the form of strips, column, circles, sheets, ovals, squares or other forms suitable for the particular assay. The first portion 36 and the second portion 38 of the binder support medium 30 can be on the same dimensional material, or can be on separate dimensional materials. For example, the two portions can be on the same strip of membrane, or the first portion 36 can be on one membrane strip, and the second portion 38 can be on a separate membrane strip, wherein the two strips are laterally juxtaposed.

The binder support medium 30 of the invention may comprise a microporous or microgranular thin layer chromatography substrate. In general, the materials can be inert and generally not react physically or chemically with any of the liquid sample 2 components, reagents, labels, buffers or reaction products.

Thin layer chromatographic substrate materials particularly suitable for use as the biner support medium can include granular thin layer chromatographic materials such as silica or microgranular cellulose. For example, non-granular microporous materials, including microporous cellulose esters, for example, esters of cellulose with an aliphatic carboxylic acid, such as an alkane carboxylic acid, having from 1 to 7 carbon atoms, e.g., acetic acid, propionic acid, or any of the butyric acids or valeric acids, can be provided. Alternatively, microporous materials made from nitrocellulose, by which term any nitric acid ester of cellulose is intended, can be provided. Suitable materials include nitrocellulose in combination with any of the said carboxylic acid cellulose esters. Thus, pure nitrocellulose esters can be used as consisting of an ester of cellulose having approximately 3 nitric groups per 6 carbon atoms. Type SMWP material (Millipore Corp., Bedford, Mass.) which has a pore size of 5 μm can also be used in the invention.

Natural, synthetic, or naturally occurring materials that are synthetically modified can also be used as the binder support medium including, but not limited to: cellulose materials such as paper, cellulose, and cellulose derivatives such as cellulose acetate and nitrocellulose; fiberglass; cloth, both naturally occurring (e.g., cotton) and synthetic (e.g., nylon); porous gels such as silica gel, agarose, dextran, and gelatin; porous fibrous matrixes; starch based materials, such as Sephadex™ brand cross-linked dextran chains; ceramic materials; films of polyvinyl chloride and combinations of polyvinyl chloride-silica; and the like. In one exemplary embodiment, glass fiber filter paper can be used as the binder support medium 30.

The binder support medium 30 may also be coated onto or bonded or laminated to appropriate inert support materials such as paper, glass, plastic, metal or fabrics. In one exemplary embodiment, the support material is Mylar. Such a support material not only has the effect of providing structural support to the binder support medium 30 but also prevents evaporation of reagent and solvent materials during the assay procedure. Cover plates may also be fashioned of such inert materials. Cover plates, although not required for practice of the invention, lend additional structural support and further prevent evaporation of reagent and solvent materials during the assay procedure. Such cover plates may be transparent for viewing the progression of the assay and may comprise apertures for addition of sample materials, chromatographic solvent or reagents.

The binder support medium 30 of the invention can provide at least one detection zone 32 vertically juxtaposed to the tracer pad 20, wherein an analyte-labeled receptor complex 25, or analyte 3, derivative, or analogue thereof, if present, is capable of being captured. The detection zone 32 vertically juxtaposed to the tracer pad 20 can be located on the first portion 36 of the binder support medium 30. The detection zone 32 can encompass an area less than the whole of the first portion 36 of the binder support medium 30, or can encompass the entire first portion 36 of the binder support medium 30. In certain exemplary embodiments, the first portion 36 of the binder support medium 30 can comprise more than one detection zone 32, wherein each detection zone is capable of detecting a different analyte of interest.

The detection zone 32 on the binder support medium 30 comprises an immobilized capture reagent 33, known as a binder, capable of binding the analyte-labeled receptor complex 25, rendering the complex immobile on the medium. In certain exemplary embodiments, the immobilized binder 33 may be capable of binding the analyte 3 and labeled receptor 22 in a competitive type assay. By “immobilized,” it is generally meant that the binder 33, once on the binder support medium 30, may not be capable of substantial movement to positions elsewhere within the binder support medium 30. Thus, the analyte-labeled receptor complex 25 can be trapped at the detection zone 32 through the binding of the binder 33 to the complex 25.

The immobilized binder 33 of the invention can include any moiety or compound capable of binding the analyte-labeled receptor complex 25, analyte 3, labeled receptor 22, or similar detectable complex contemplated in the type of assay employed. For example, the binder 33 can consist of any ligand capable of binding the analyte-labeled receptor complex 25. One example can include the use of an antibody as the binder 33, wherein the antibody is capable of binding directly to the analyte 3 in the analyte-labeled receptor complex 25. Specific binding reagents useful with the invention are known by one of ordinary skill in the art and are generally readily identifiable.

Because the binder support medium 30 of the device is preferably chemically inert, it can be activated at the detection zone 32 where it is desired to immobilize a specific binding reagent 33. Various methods can render the binder reagent 33 immobilized according to the particular chemical nature of the binder support medium 30 material. The immobilized binder 33 may be supported on the binder support medium 30 in a manner which immobilizes the binder 33. The binder 33 can be immobilized to the binder support medium 30 directly or indirectly. Direct attachment methods can include adsorption, absorption and covalent binding such as by use of (i) a cyanogen halide, e.g., cyanogen bromide or (ii) by use of glutaraldehyde. Other methods may include treatment with Schiff bases and borohydride for reduction of aldehydic, carbonyl and amino groups. DNA, RNA and certain antigens may be immobilized against solvent transport by baking onto the binder support medium 30.

Depending on the assay, it may be preferred, however, to retain or immobilize the binder reagent 33 on the binder support medium 30 material indirectly through the use of insoluble microparticles to which the binder reagent 33 has been attached. The methods of attaching an reagent to the microparticles encompass both covalent and non-covalent mechanisms including adherence, absorption, or adsorption. Microparticles are generally known by one of ordinary skill in the art. For example, microparticles can be selected from any suitable type of particulate material composed or polystyrene, polymethylacrylate, polyacrylamide, polypropylene, latex, polytetrafluoroethylene, polyacrylonitrile, polycarbonate, glass or similar material.

The binder 33 can be deposited singly or in various combinations on or in the detection zone 32 of the binder support medium 30 in a variety of configurations to produce different detection or measurement formats. Such configurations and measurement formats are generally known by one of ordinary skill in the art.

The binder support medium 30 may further comprise a control zone 34 capable of conveying an indication to the user that the liquid sample 2 has flowed to the control zone 34, and thus through the detection zone 32 since the detection zone precedes the control zone 34. For example, the control zone 34 can comprise an anhydrous reagent that, when moistened, produces a color change or color formation, such as anhydrous copper sulphate which will turn blue when moistened by an aqueous sample.

Alternatively, the control zone may indicate that the performance of the assay was within acceptable physiological conditions. For example, the control zone 34 can comprise immobilized analyte 3 that will react with excess labeled receptor 22 that does not bind the analyte of interest 3, indicating that the conditions of the assay are within a physiological range that allows binding.

In addition, the control zone can comprise an immobilized ligand that is not the analyte of interest. The immobilized ligand may be capable of binding to a control labeled receptor. Preferably, the control labeled receptor is not capable of binding to the analyte of interest, or to a immobilized binder located in the detection zone. An exemplary embodiment of a strategy utilizing a control labeled receptor capable of binding to an immobilized ligand, in the control zone is described in FIG. 5.

A pH indicator dye can also be selected as a control zone reagent to respond to the pH of the liquid sample 2. For example, phenolphthalein changes from clear to intense pink upon contact with a solution having a pH range between 8.0-10.0, which is a common pH range for the assay fluids. Such a control zone indicator would reveal whether the pH conditions of the assay were within acceptable ranges, and alert the operator that the liquid sample 2 has permeated the required distance through the test device.

The control zone 34 can be located in any position on the binder control medium 30 to allow for analysis. For example, the control zone 34 can be located downstream from the detection zone 32. In alternative exemplary embodiments, the control zone 34 can be located adjacent to, or within the detection zone 32 in which the desired test result is recorded.

Following application of the liquid sample 2 to the tracer pad application point 12, the operator can allow the assay to proceed. After a short period, the operator may analyze the control zone 34 to determine if the liquid sample 2 has proceeded through the detection zone 32, and if the assay conditions were in an acceptable working range. The operator then may analyze the detection zone 32 to determine if the analyte of interest 3 is present in the liquid sample 2.

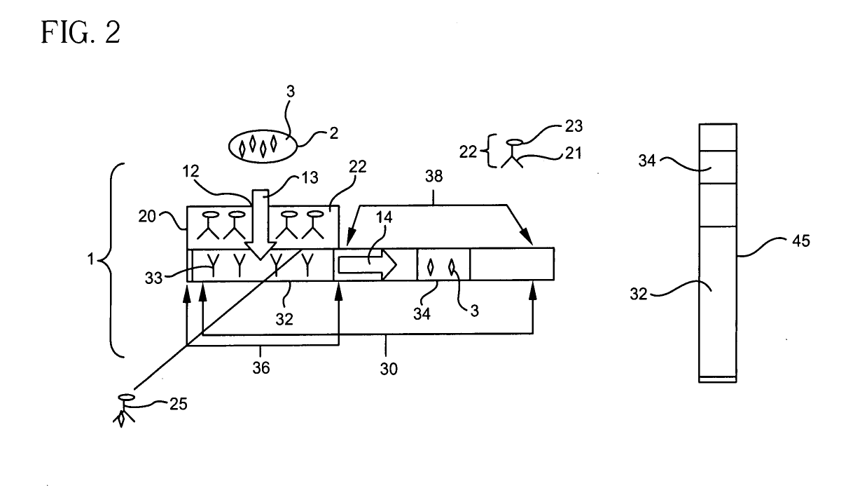

FIG. 2 illustrates an overview of the detection zone 32 of the assay 1. In this exemplary embodiment, the label 23 is one that can be visually detected by the eye of the operator, and the operator can analyze the detection zone 32 for the presence or absence of the analyte 3 by viewing the detection zone 32 on the side 45 of the first portion 36 of the binder support medium 30 that is not covered by the vertically juxtaposed tracer pad 20. In the exemplary embodiment, a positive result will be visually indicated by the presence of a threshold amount of a visually detectable labeled receptor 22 in the detection zone 32. If the label 23 is one that can be detected by a machine, then the operator can place the device 1 on a machine for analysis.

FIG. 3 is similar to FIG. 1, wherein the assay type illustrated is a competitive type assay. In a competitive assay the receptor 21 portion of the labeled receptor 22 is generally not capable of binding to the analyte of interest 3. The receptor 22 may be the analyte 3 or an appropriate analogue thereof. The term “appropriate analogue” generally means that the analogue of the receptor 21 is also specifically bound by the immobilized binder 33 of the analyte 3 in the detection zone 32. The label 23 of the labeled receptor 22 can be coupled to a receptor 21 which is competitive with the analyte of interest 3. Both the analyte 3 from the liquid sample 2 and the competitor labeled receptor 22 progress with the flow 13 of the liquid sample 2 to the detection zone 32. Both the analyte 3 and the labeled receptor 22 then compete with each other in the binding of the immobilized binder reagent 33 positioned on the detection zone 32. The unlabeled analyte 3 thus is able to reduce the quantity of labeled receptor 22 captured in the detection zone 32. This reduction in retention of the labeled receptor 22 becomes a measure of the presence of the analyte 3 in the liquid sample 2. The control zone 34 may comprise an anhydrous reagent that changes color upon contact with the laterally flowing liquid sample 2.

FIG. 4 is similar to FIG. 3, wherein the assay type illustrated is a competitive type assay. In this exemplary competitive assay embodiment, the receptor 21 portion of the labeled receptor 22 is capable of binding to the analyte of interest 3. The receptor 21 may be an antibody or other ligand capable of binding the analyte of interest 3. However, unlike in FIG. 3, the immobilized binder 33 is not capable of binding to the analyte 3. Instead, the immobilized binder 33 is capable of binding to the receptor 21 of the labeled receptor 22. As the liquid sample 2 encounters the labeled receptor 22, the labeled receptor 22 may bind the analyte 3 if present, forming an analyte-labeled receptor complex 25. Both the analyte-labeled receptor complex 25 and unbound labeled receptor 22 progress with the flow 13 of the liquid sample 2 to the detection zone 32. The immobilized binder reagent 33 may be the analyte of interest 3, homologue, or derivative thereof, capable of binding to the unbound labeled receptor 22. The analyte of interest 3 in the liquid sample 2 thus competes with the immobilized binder 33 for the labeled receptor 22. The analyte 3 thus is able to reduce the quantity of labeled receptor 22 captured in the detection zone 32. This reduction in retention of the labeled receptor 22 becomes a measure of the presence of the analyte 3 in the liquid sample 2. The control zone 34 may contain a ligand 37 capable of binding to the analyte-labeled receptor complex 25.

FIG. 5 is similar to FIG. 1, wherein the tracer pad 20 further comprises a control labeled receptor 28 capable of binding to a ligand 5 immobilized in the control zone 34. Such a control labeled receptor 28 provides a mechanism to analyze the binding conditions of the assay, wherein the binding of the control labeled receptor 28 to the immobilized ligand 5 in the control zone indicates that proper binding conditions are present in the assay.

The label 281 of the control labeled receptor 28 may provide a different visual indicator than the label 23 of the labeled receptor 22. For example, the label 281 of the control labeled receptor 28 may produce a different color than the label 23 of the labeled receptor 22. Alternatively, the label 281 of the labeled receptor 28 may be the same label as the label 23 of the labeled receptor 22. In addition, the receptor 282 of the additional label receptor 28 may be specific for an immobilized ligand 5 contained in the control zone 34. Preferably, the receptor 282 of the control labeled receptor 28 is not capable of binding the analyte of interest 3, or an immobilized binder 33 contained in the detection zone 34.

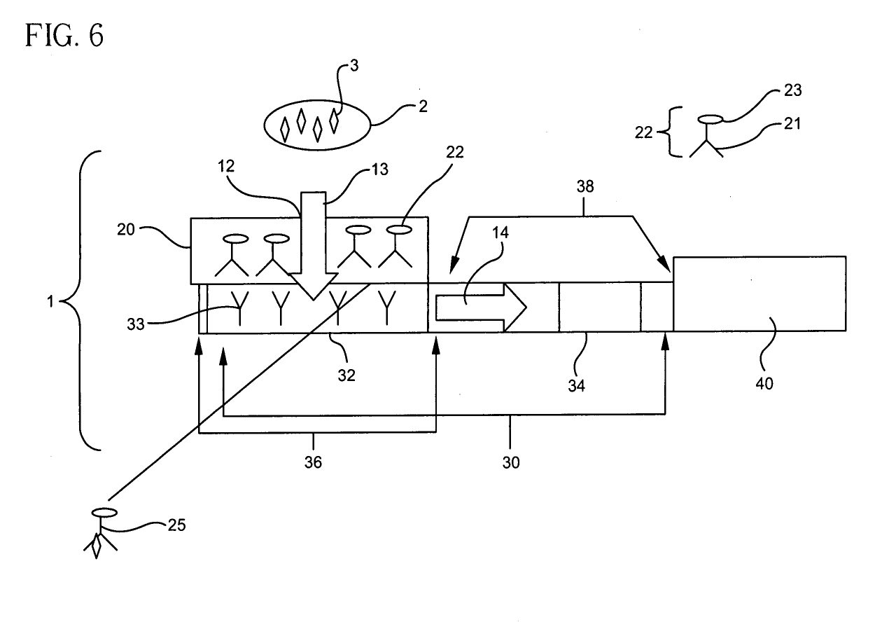

FIG. 6 is similar to FIG. 1, with the added element of a sump 40 laterally juxtaposed with the binder support medium 30. The liquid sample 2, following contact with the detection zone 32, is capable of lateral flow 14 on the binder support medium 30 from the first portion 36 of the binder support medium 30 to the second portion 38 of the binder support medium 30, and then to the sump 40. The sump 40 can act as a collection point for the liquid sample 2, wicking or absorbing the liquid sample 2 as it reaches the end of the second portion 38 of the binder support medium 30. The sump 40 can prevent the liquid sample 2 from flowing back into the first portion 36 of the binder support medium 30. The sump 40 can have absorbent capacity sufficient to contain all liquid volumes used during the assay. The sump 40 can be comprised of any material capable of absorbing excess liquid. Examples of such materials include cotton fiber, tissue, and absorbent paper, such as Whatman paper. Other examples of suitable absorbent materials capable of acting as a sump 40 are generally known by one of ordinary skill in the art.

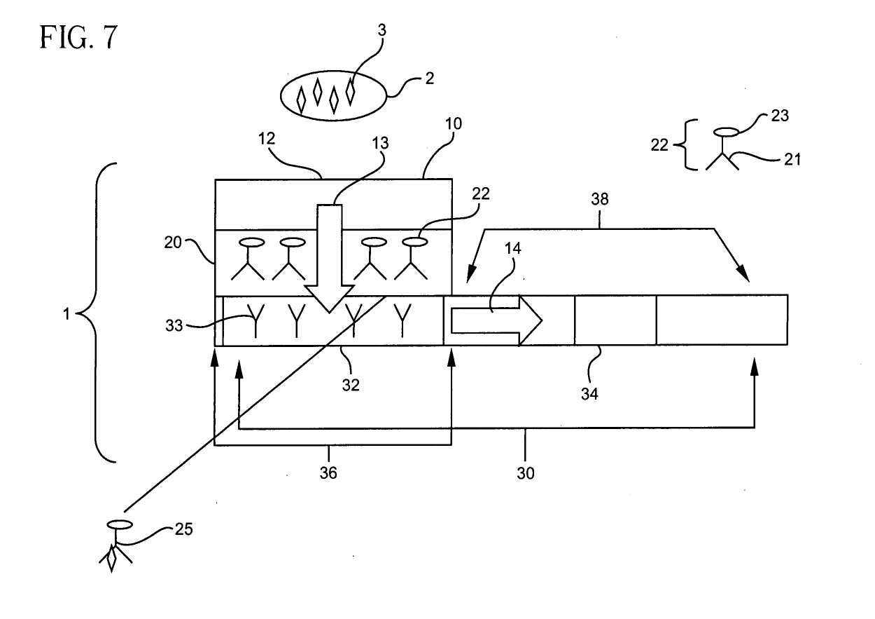

FIG. 7 is similar to FIG. 1, with the added element of a sample pad 10 vertically juxtaposed with the tracer pad 20. The liquid sample 2 can be applied at the liquid sample application site 12 on any area of the sample pad 10. The liquid sample 2 vertically flows 13 through the sample pad 10 to the vertically juxtaposed tracer pad 20, wherein it can interact with the analyte specific labeled receptor 22 contained therein.

The sample pad 10 can provide a receiving or collection point on the device for the application of the liquid sample 2. The sample pad 10 can be any porous material capable of receiving a liquid sample. The sample pad 10 of the invention can be made from any hydrophilic porous or fibrous material capable of absorbing liquid rapidly. For example, porous plastics material, such as polypropylene, polyethylene, polyvinylidene fluoride, ethylene vinylacetate, acrylonitrile and polytetrafluoro-ethylene can be used as a sample pad 10. Materials such as cellulosic materials including nitro-cellulose, acrylic fibers such as non-woven spun-laced acrylic fiber (i.e., New Merge from DuPont) or HDK material (from HDK Industries, Inc.), glass, fiber, filter paper or pads, desiccated paper, paper pulp, fabric, and the like can also be used. The material selected for use as a sample pad 10 may also be chosen for its compatibility with the analyte 3 and assay reagents. For example, glass fiber filter paper may be utilized as a sample pad 10 material for use in a human chorionic gonadotropin (hCG) assay device.

It certain instances, it may be advantageous to pre-treat the sample pad 10 with a surface-active agent during manufacture in order to reduce any associated hydrophobicity, and enhance the ability of the sample pad 10 to take up a liquid sample 2 rapidly and efficiently. In one exemplary embodiment, the sample pad 10 can be constructed from any material that may be capable of absorbing an aqueous solution. In another exemplary embodiment, the sample pad 10 can be comprised of any material from which the liquid sample 2 can flow trough to the tracer pad 20. The material comprising the sample pad 10 can be chosen such that the sample pad 10 can be saturated with the liquid sample 2 within a matter of seconds.

In additional exemplary embodiments, the functions of the sample pad 10 may further include, for example: pH control/modification and/or specific gravity control/modification of the liquid sample 2 applied, removal or alteration of components of the liquid sample 2 which may interfere or cause non-specific binding in the assay, or to direct and control liquid sample flow to the tracer pad 20.

In certain exemplary embodiments, the sample pad 10 may include components that assist in the transfer of the liquid sample 2 through the assay device. For example, when small quantities of viscous liquid sample 2 are applied to the sample pad 10, it may be necessary to employ a wicking solution, preferably a buffered wicking solution, to carry the viscous liquid sample 2 from the sample pad 10 and through the assay device of the invention. When an aqueous liquid sample 2 is used, a wicking solution generally is not necessary but can be used to improve flow characteristics or adjust the pH of the liquid sample 2. The wicking solution can typically have a pH range from about 5.5 to about 10.5, and more particularly from about 6.5 to about 9.5. The pH is selected to maintain a significant level of binding affinity between the specific binding members in a binding assay. When the label 23 component of the label receptor 22 is an enzyme, however, the pH also must be selected to maintain significant enzyme activity for color development in enzymatic indicator production systems. Illustrative buffers include phosphate, carbonate, barbital, diethylamine, tris, 2-amino-2-methyl-1-propanol and the like. In one exemplary embodiment, the wicking solution can be contained on the sample pad 10. In another exemplary embodiment, the wicking solution and the liquid sample 2 can be combined prior to contacting the sample pad 10. In an alternative exemplary embodiment, the wicking solution and the liquid sample 2 can be applied sequentially to the sample pad 10.

In another exemplary embodiment, the sample pad 10 may also incorporate reagents useful to avoid cross-reactivity with non-target analytes that may exist in the liquid sample 2 and/or to condition the liquid sample 2. These reagents may include, for example, non-hCG blockers, anti-RBC reagents, Tris-based buffers, and EDTA, among others. When the use of whole blood is contemplated, anti-RBC reagents are frequently utilized. The sample pad 10 may also incorporate other reagents such as ancillary specific binding members, liquid sample pretreatment reagents, and detection reagents.

In exemplary embodiments, the sample pad 10 can be constructed to act as a filter for cellular components, hormones, particulate, and other certain substances that may be present in the liquid sample 2. The filtering aspect allows an analyte of interest 3 to migrate through the device in a controlled fashion with fewer interfering substances than if the filtering aspect was not present. The filtering aspect can provide for a test having a higher probability of success and accuracy. The sample pad 10 can further be modified by the addition of a filtration mechanism. The filtration mechanism can include any filter or trapping device used to remove particles above a certain size from the liquid sample 2. For example, the filtration mechanism can be used to remove red blood cells from a sample of whole blood, such that plasma is the fluid received by the tracer pad 20.

In one exemplary embodiment, the sample pad 10 further comprises an analyte of interest 3, analyte homologue, or derivative thereof for use in a competitive assay.

Once introduced onto the sample pad 10 at the sample application site 12, the liquid sample 2 can thereafter permeate freely from the sample pad 10 onto the vertically juxtaposed tracer pad 20. The sample pad 10 can be in direct vertical fluid flow contact with the tracer pad 20, such that the liquid sample 2 can pass or migrate from the sample pad 10 to the tracer pad 20 via vertical flow 13. Fluid flow contact can include physical contact of the sample pad 10 to the tracer pad 20, as well as the separation of the sample pad 10 from the tracer pad 20 by an intervening space or additional material which still allows vertical fluid flow 13 between the sample pad 10 and tracer pad 20. In one exemplary embodiment, substantially all of the sample pad 10 can overlap the tracer pad 20 to enable the liquid sample 2 to vertically pass through substantially any part of the sample pad to the tracer pad.

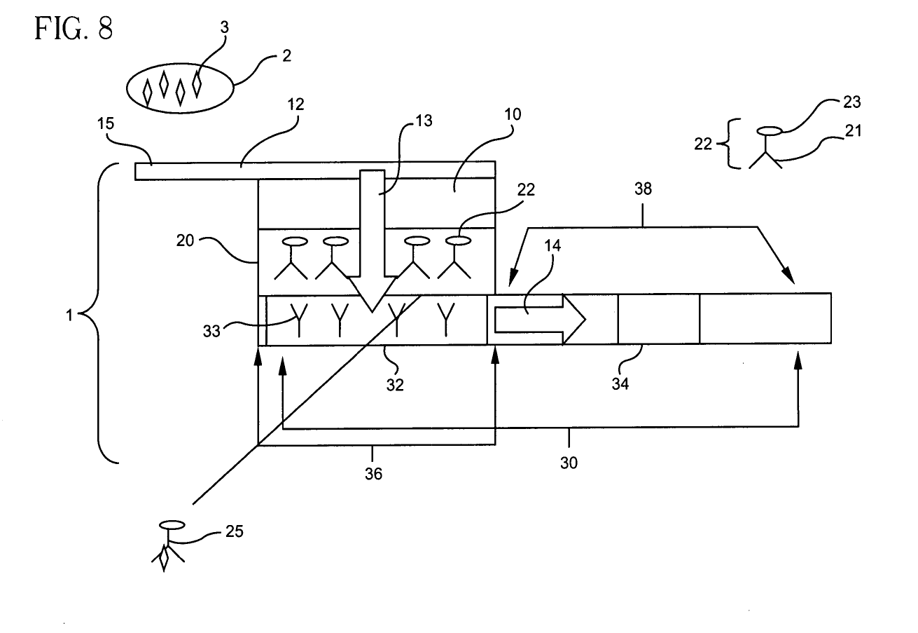

FIG. 8 is similar to FIG. 7, wherein the sample pad 10 further comprises an additional sample application member 15. The sample application member 15 can be vertically juxtaposed or laterally juxtaposed to the sample pad 10, so long as the sample application member 15 is positioned in fluid-flow contact with the sample pad 10. The fluid flow contact can comprise an overlapping, abutting or interlaced type of contact, wherein liquid sample that is applied to the sample application member 15 is able to flow to the tracer pad 20.

The sample application member 15 can be comprised of a material that readily absorbs any of a variety of liquid samples and remains robust in physical form. For example, the sample application member 15 can be comprised of a material such as white bonded polyester fiber. In exemplary embodiments, the sample application member 15 may be treated with a hydrophilic finishing. The sample application member 15 may also comprise similar reagents and be comprised of similar materials to those utilized in exemplary sample pads 10.

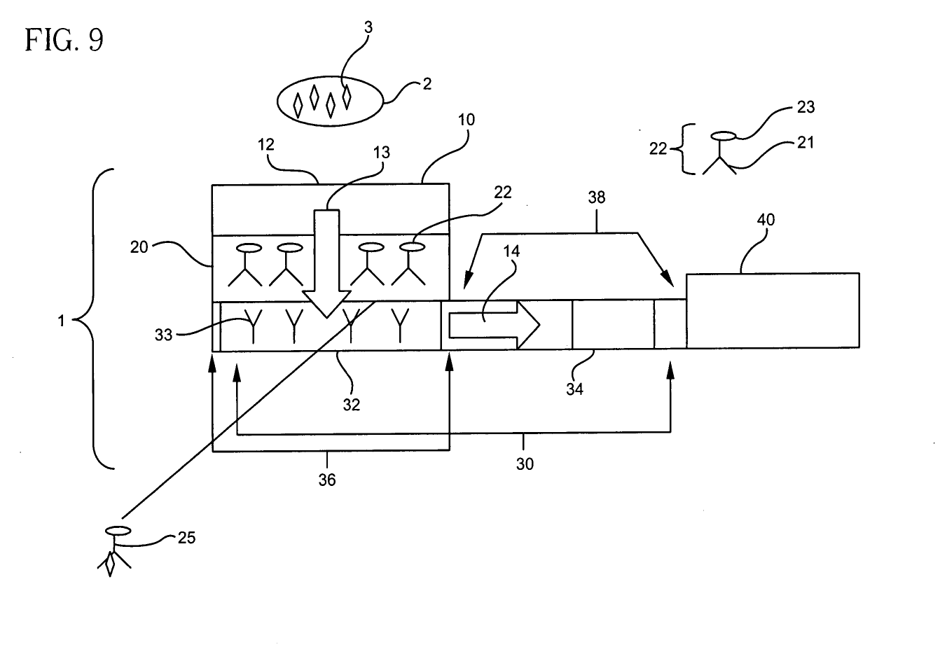

FIG. 9 is similar to FIG. 7, with the added element of a sump 40 laterally juxtaposed to the binder support medium 30.

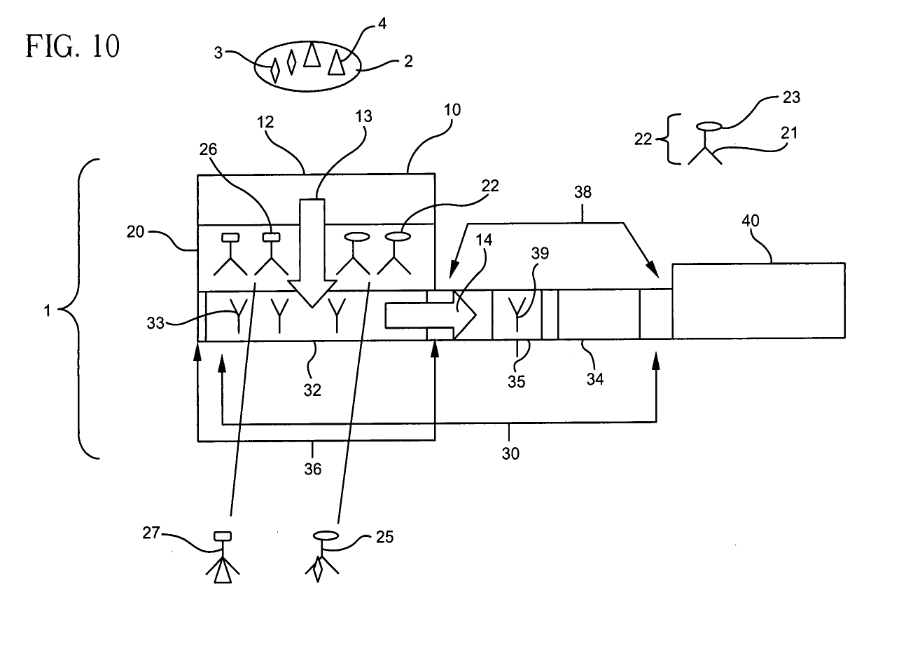

FIG. 10 is similar to FIG. 9, wherein the second portion 38 of the binders support medium further comprises an additional detection zone 35. The tracer pad 20 can comprise more than one labeled receptor specific for more than one specific analyte, wherein the labeled receptors have different detectable characteristics (e.g., different colors) such that one labeled receptor-analyte complex can be differentiated from another labeled receptor-analyte complex in the associated detection zones. For example, the tracer pad can comprise a first labeled receptor 22 that binds a first analyte 3, forming a first analyte-labeled receptor complex 25 that can be bound by a specific immobilized binder 33 in a first detection zone 32, and a second labeled receptor 26 that binds a second analyte 4, forming a second analyte-labeled receptor complex 27 that can be bound by a specific immobilized binder 39 in a second detection zone 35.

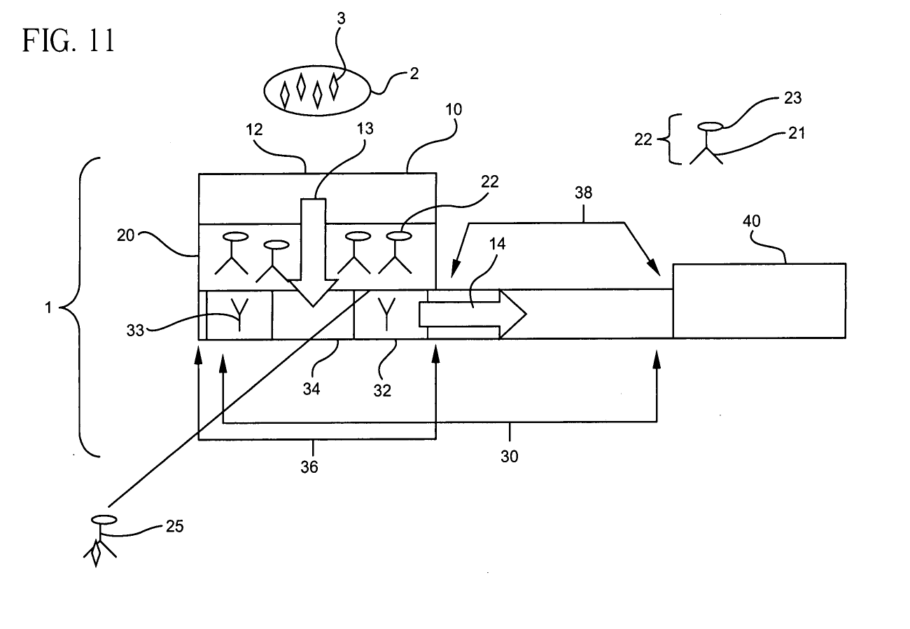

FIG. 11 is similar to FIG. 9, wherein the first portion 36 of the binder support medium 30 further comprises a control zone 34. The control zone 34 can be located adjacent to, or within the detection zone 32. Techniques for including a control zone 34 in the area of a detection zone 32 are known by one of ordinary skill in the art.

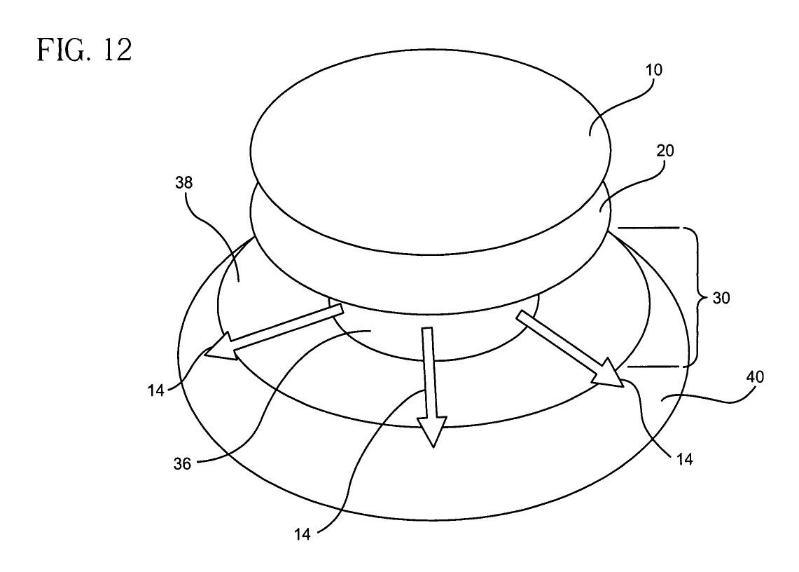

FIG. 12 illustrates an alternate exemplary embodiment of an immunoassay device 1, wherein the sump 40 surrounds the binder support medium 30. This arrangement can allow for multi-directional lateral flow 14 of the liquid sample 2 from the first portion 36 of the binder support medium 30 to the second portion 38 of the binder support medium 30, and then to the sump 40, which surrounds the binder support medium 30 and is in lateral contact with the binder support medium 30.

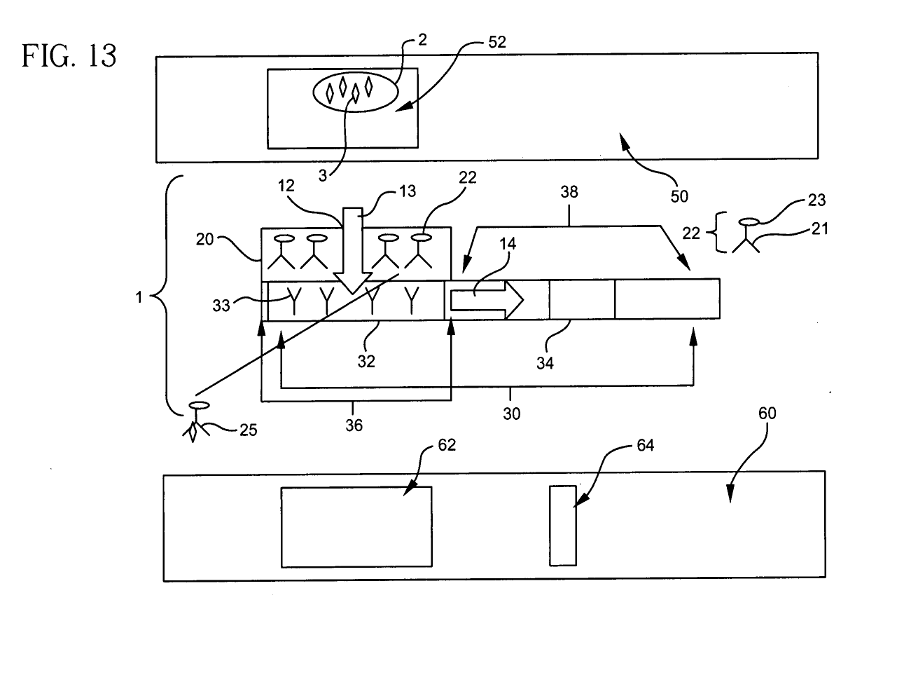

FIG. 13 is a schematic illustrating an exemplary embodiment wherein the device 1 is placed between an upper housing 50 and a lower housing 60. The upper housing 50 unit contains an aperture 52 for access to the tracer pad 20 application zone 12. The lower housing 60 contains two apertures, an aperture 62 for visual access to the detection zone 32 and an aperture 64 for visual access to the control zone 34.

In one exemplary embodiment, the size of the upper housing 50 and lower housing 60 allows for convenient handling and packaging of the device. The upper housing 50 and lower housing 60 of the invention can be made of plastic, glass or other suitably rigid material. The upper housing 50 and lower housing 60 can serve other functions as well, including providing a handle or displaying information such as bar codes, fluorescent marks, or colored marks which can aid in the calibration of the assay. The upper housing 50 and lower housing 60 material can be in sheet or roll form, and can be manufactured from an opaque plastic sheet material of appropriate color, thickness, and rigidity.

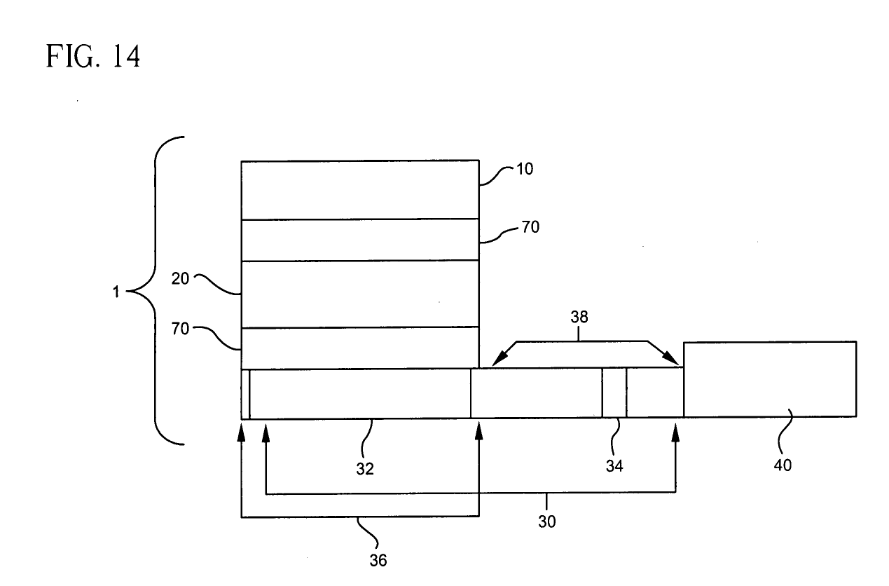

FIG. 14 is a schematic of an exemplary embodiment illustrating the inclusion of optional spacers 70. In this example, the spacers 70 are placed between a sample pad 10 and a tracer pad 20, and the tracer pad 20 and a binder support medium 30. The spacer 70 can serve as a mechanism to control the rate of flow of the liquid sample 2 from the sample pad 10 to the tracer pad 20 or the tracer pad 20 to the binder support medium 30. Such flow regulation is preferred when an extended incubation period is desired for the reaction of the liquid sample 2 and the reagent(s) in the tracer pad 20. Alternatively, such a layer can comprise an additional assay reagent(s) which is preferably isolated from the tracer pad 20 reagents until the liquid sample 2 is added, or it can serve to prevent un-reacted assay reagents from passing to the binder support medium 30.

In exemplary embodiments, the spacer 70 can be constructed to act as a filter for cellular components, hormones, particulate, and other certain substances that may be present in the liquid sample 2. The filtering aspect allows an analyte of interest 3 to migrate through the device in a controlled fashion with fewer interfering substances than if the filtering aspect was not present. The filtering aspect can provide for a test having a higher probability of success and accuracy. The spacer 70 can further be modified by the addition of a filtration means. The spacer 70 can include any filter or trapping device used to remove particles above a certain size from the liquid sample 2. For example, the spacer 70 can be used to remove red blood cells from a sample of whole blood, such that plasma is the fluid received by the tracer pad 20.

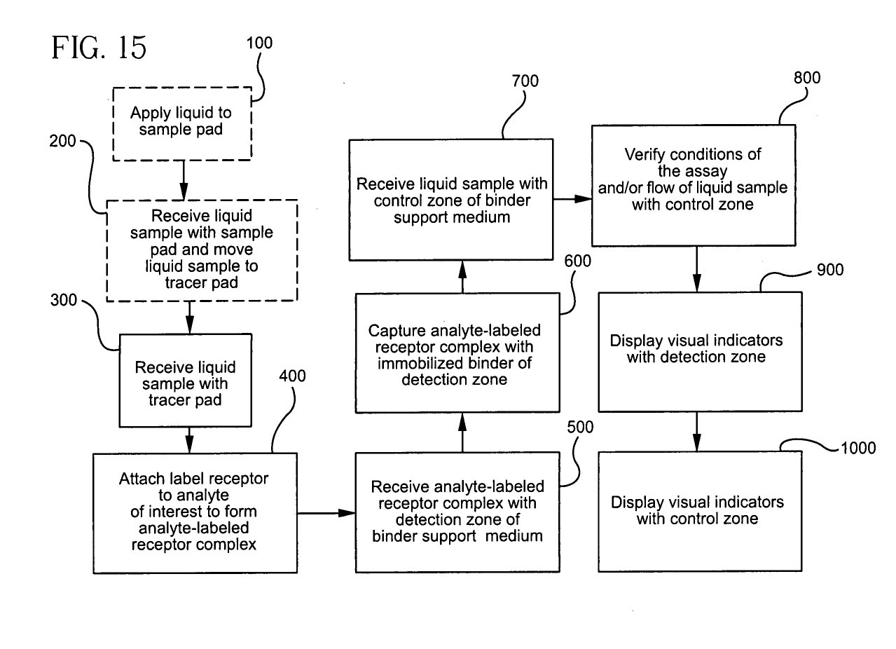

FIG. 15 is a flow chart illustrating a method for performing an analysis for the presence or absence of an analyte of interest in a liquid sample. The illustrative flow chart describes the exemplary embodiment of the performance of a sandwich type assay utilizing the device of the present invention. The flow chart described may be modified for alternative type assays such as competitive type assays.

In optional step 100, initiate the process of analyzing the presence or absence of an analyte of interest in a liquid sample by applying a liquid sample to the optional sample pad of the assay device. In step 200, the sample pad receives the liquid sample applied thereto, and moves through vertical flow to the tracer pad. In step 300, if a sample pad is not present in the assay device, initiate the process by applying the liquid sample to the tracer pad, where the liquid sample is received. If a sample pad is present in the device, the tracer pad receives the liquid sample from the sample pad. In step 400, attach the labeled receptor capable of binding to an analyte of interest to the analyte of interest, if present in the liquid sample, to form an analyte-labeled receptor. In step 500, receive the analyte-labeled receptor complex with detection zone of the binder support medium through vertical flow from the tracer pad, wherein the detection zone is vertically juxtaposed with the tracer pad. In step 600, capture the analyte-labeled complex with the immobilized binder of the detection zone. In step 700, receive the liquid sample with the control zone of the binder support medium. In step 800, verify the conditions of the assay and/or flow of liquid sample with the control zone. In step 900, display visual indicators with the detection zone. In step 1000, display visual indicators with the control zone.

The process allows the user to determine if the liquid sample contains an analyte of interest. Analyte-receptor labeled complexes that are captured by the immobilized binder can be capable of detection, either visually by the eye of the operator or via a measuring device. After an appropriate time, the operator can analyze the control zone visually or with the aid of a detection device to ascertain whether the assay has been completed, and then can ascertain the result of the assay by observing the detection zone. Certain steps in the method described above must naturally precede others for the invention to function as described. However, the invention is not limited to the order of the steps described if such order or sequence does not alter the functionality of the invention. That is, it is recognized that some steps may be performed before or after or in parallel with other steps without departing from the scope and spirit of the invention.

Kits

The invention further provides kits for carrying out immuno-assays utilizing the device as described herein. For example, a kit according to the invention can comprise the assay device with its incorporated reagents as well as a wicking solution and/or test sample pretreatment reagents. Other assay components known to one of ordinary skill in the art, such as buffers, stabilizers, detergents, bacteria inhibiting agents and the like can also be present in the kit. In addition, the kit can contain packaging materials and directions on how to use the device.

It should be understood that the foregoing relates only to illustrate the embodiments of the invention, and that numerous changes may be made therein without departing from the scope and spirit of the invention as defined by the following claims.

### EXAMPLES

The following example illustrates, but is not intended to limit the invention.

### Example 1

a. Construction of Immunoassay Device

Immunoassay devices were constructed according to the present invention. In general, a tracer pad was placed directly on top of a binder support medium. The tracer pad was placed so that only a portion of the binder support medium was in direct contact with the tracer pad, leaving a portion of the binder support medium uncovered by the tracer pad. The tracer pad was placed directly over the portion of the binder support medium containing the detection zone. In addition, a sump pad was placed at the end of the binder support medium, allowing excess sample to flow from the binder support medium to the adjacent sump.

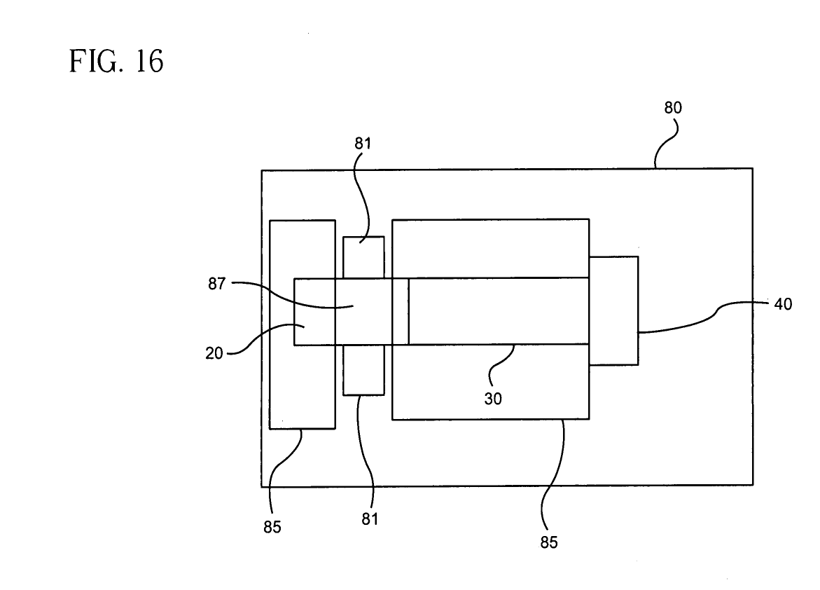

FIG. 16 is an overview of a representative schematic of the constructed immunoassay devices. Two immunoassay devices for the detection of a Respiratory Syncytial Virus (RSV) antigen were constructed. A nitrocellulose binder support medium 30 measuring 5.5×25 mm was removed from a Becton-Dickenson EZ RSV Kit (Catalog #256030). The nitrocellulose binder support medium 30 comprised a monoclonal antibody directed against a Respiratory Syncytial Virus (RSV) in the detection zone as an immobilized binder. The nitrocellulose strip was placed on a square piece of parafilm 80, and the location of the detection zone on the binder support medium was marked 81 on the parafilm on both sides of the binder support medium 30.

A tracer pad 20 was removed from a Becton-Dickenson EZ RSV Kit (Catalog #256030) and cut to a dimension of 5.5×7 mm. The tracer pad 20 contained a labeled receptor. The receptor was a monoclonal antibody directed against the RSV antigen. The receptor was conjugated to a colloidal gold label. The tracer pad 20 was placed directly on top of a portion of the binder support medium 30, directly over the detection zone. A sump 40 was placed adjacent to the end of the binder support medium 30 that was not covered by the tracer pad. The tracer pad 20 and binder support medium 30 were then covered with G & L Clearbiax-polypropylene tape 85, except for a 4.5×6 mm center opening 87 on the tracer pad 20. The steps of construction were identical for the construction of the second immunoassay device.

b. Detection of RSV Antigen

75 ul of a liquid sample containing RSV antigen 407909jaa was applied to the tracer pad at the 4.5×6 mm center opening on the tracer pad of one of the constructed immunoassays. The assay was allowed to proceed. The assembly was then turned over in order to analyze the detection zone. The detection zone indicated a positive result for the presence of the RSV antigen.

Likewise, 75 ul of a liquid sample lacking RSV antigen 407909jaa was applied to the tracer pad at the 4.5×6 mm center opening on the tracer pad of the other constructed immunoassay. The assay was allowed to proceed for 15 minutes. The assembly was then turned over in order to analyze the detection zone. The detection zone indicated a negative result for the presence of the RSV antigen.

We claim:

1) An immunoassay device for analyzing an analyte of interest in a liquid sample comprising:
a) a tracer pad comprising a labeled receptor; and
b) a binder support medium comprising at least one detection zone for detecting the analyte of interest, the binder support medium comprising at least a first portion and a second portion;
wherein the first portion of the binder support medium is vertically juxtaposed to the tracer pad, the first portion comprising at least one detection zone for detecting the analyte of interest, and the second portion of the binder support medium is not vertically juxtaposed with the tracer pad, the second portion providing lateral fluid flow access for the liquid sample away from the first portion.

2) The device of claim 1, wherein the second portion of the binder support medium further comprises a control zone for determining the viability of the assay.

3) The device of claim 1, wherein the second portion of the binder support medium further comprises a detection zone for detecting an analyte of interest.

4) The device of claim 1, wherein the first portion of the binder support medium further comprises a control zone for determining the viability of the assay.

5) The device of claim 1, further comprising at least one spacer pad vertically juxtaposed between the tracer pad and the binder support medium.

6) The device of claim 5, further comprising a sample pad application member, wherein the sample pad application member is in fluid flow contact with the sample pad.

7) The device of claim 1 or 6, further comprising a spacer pad vertically juxtaposed between the tracer pad and the binder support medium.

8) The device of claim 1, further comprising a sump, wherein the sump is laterally juxtaposed to the binder support medium.

9) The device of claim 8, wherein the sump surrounds the binder support medium.

10) The device of claim 1, further comprising an upper housing and a lower housing.

11) An immunoassay device for analyzing an analyte in a liquid sample comprising:
a) a sample pad capable of accepting a liquid sample containing an analyte of interest;
b) a tracer pad comprising a labeled receptor, wherein the tracer pad is vertically juxtaposed with the sample pad; and
c) a binder support medium comprising at least one detection zone, the binder support medium comprising at least a first portion and a second portion;
wherein the first portion of the binder support medium is vertically juxtaposed to the tracer pad, the first portion comprising at least one detection zone for detecting the analyte of interest, and the second portion of the binder support medium is not vertically juxtaposed with the tracer pad, the second portion providing lateral fluid flow access for the liquid sample away from the first portion.

12) The device of claim 11, wherein the second portion of the binder support medium further comprises a control zone for determining the viability of the assay.

13) The device of claim 11, wherein the second portion of the binder support medium further comprises a detection zone for detecting an analyte of interest.

14) The device of claim 11, wherein the first portion of the binder support medium further comprises a control zone for determining the viability of the assay.

15) The device of claim 11, further comprising at least one spacer pad vertically juxtaposed between the tracer pad and the binder support medium.

16) The device of claim 11 or 15, further comprising a spacer pad vertically juxtaposed between the tracer pad and the binder support medium.

17) The device of claim 16, wherein the sump surrounds the binder support medium.

18) The device of claim 11, further comprising an upper housing and a lower housing.

19) The device of claim 11, further comprising a sample pad application member, wherein the sample pad application member is in fluid flow contact with the sample pad.

20) An immunoassay device 1 for analyzing an analyte in a liquid sample comprising:
a) a sample pad capable of accepting a liquid sample containing an analyte of interest;
b) a tracer pad comprising a labeled receptor, wherein the tracer pad is vertically juxtaposed with the sample pad;
c) a binder support medium comprising at least one detection zone, the binder support medium comprising at least a first portion and a second portion; and
d) a sump, wherein the sump is laterally juxtaposed to the binder support medium;
wherein the first portion of the binder support medium is vertically juxtaposed to the tracer pad, the first portion comprising at least one detection zone for detecting the analyte of interest, and the second portion of the binder support medium is not vertically juxtaposed with the tracer pad, the second portion providing lateral fluid flow access for the liquid sample away from the first portion.

21) The device of claim 20, wherein the second portion of the binder support medium further comprises a control zone for determining the validity of the assay.

22) The device of claim 20, wherein the second portion of the binder support medium further comprises a detection zone for detecting an analyte of interest.

23) The device of claim 20, wherein the first portion of the binder support medium further comprises a control zone for detecting an analyte of interest.

24) The device of claim 20, further comprising at least one spacer pad vertically juxtaposed between the tracer pad and the binder support medium.

25) The device of claim 20 or 24, further comprising a spacer pad vertically juxtaposed between the tracer pad and the binder support medium.

26) The device of claim 20, wherein the sump surrounds the binder support medium.

27) The device of claim 20, further comprising an upper housing and a lower housing.

28) The device of claim 20, further comprising a sample pad application member, wherein the sample pad application member is in fluid flow contact with the sample pad.

29) A method for determining the presence or absence of an analyte of interest in a liquid sample comprising:
a) applying a liquid sample to an assay device, wherein the liquid sample flows:
i. vertically to a first portion of a binder support medium from a tracer pad comprising a labeled receptor, the first portion of the binder support medium being vertically juxtaposed to the tracer pad, and the first portion comprising at least one detection zone for detecting an analyte of interest; and,
ii. laterally from the first portion of the binder support medium to the second portion of the binder support medium, wherein the second portion of the binder support medium is not vertically juxtaposed with the tracer pad; and
b) determining the presence or absence of the analyte in the liquid sample by analyzing the detection zone.
